(19) United States
(12) Patent Application Publication
(10) Pub. No.: US 2026/0008844 A1
(43) Pub. Date: Jan. 8, 2026

(54) USE OF ANTI-MARCO ANTIBODY WITH A CHECKPOINT BLOCKADE ANTIBODY FOR THE TREATMENT OF CANCER

(71) Applicant: H. LEE MOFFITT CANCER CENTER AND RESEARCH INSTITUTE, INC., Tampa, FL (US)

(72) Inventors: James MULÉ, Lutz, FL (US); Hidenori TAKAHASHI, Tampa, FL (US)

(21) Appl. No.: 19/111,862

(22) PCT Filed: Sep. 15, 2023
(86) PCT No.: PCT/US2023/032856

§ 371 Date: Mar. 14, 2025

Related U.S. Application Data
(60) Provisional application No. 63/407,118, filed on Sep. 15, 2022.

(51) Int. Cl. C07K 16/28; A61K 39/00; A61P 35/00; A61P 37/04

(52) U.S. Cl. CPC C07K 16/28; A61P 35/00; A61P 37/04; C07K 16/2818; C07K 16/2827; A61K 2039/507; A61K 2039/54

(57) ABSTRACT

Disclosed are combination therapies comprising an anti-macrophage receptor with collagenous structure (MARCO) antibody and an immune checkpoint inhibitor and methods of using the same to treat cancer.

### I. CROSS REFERENCE TO RELATED APPLICATIONS

This application claims the benefit of U.S. Application No. 63/407,118, filed on Sep. 15, 2022, which is incorporated herein by reference in its entirety.

### II. BACKGROUND

Anti-CTLA4 Ab therapy is one of the standard therapies for various cancer, especially in human melanoma. However, the clinical efficacy of this single anti-CTLA4 therapy is known to be limited and only observed in less than 30% of the patients. What is needed are new treatment therapies or treatment regimens that can increase the efficacy of anti-CTLA-4 in the treatment of cancer.

### III. SUMMARY

Disclosed are combination therapies comprising an anti-macrophage receptor with collagenous structure (MARCO) antibody and an immune checkpoint inhibitor and methods of using the same.

In one aspect, disclosed herein are combination therapies comprising an anti-macrophage receptor with collagenous structure (MARCO) antibody (such as, for example, ED31, PY265, F3, Ab231046, AF7586, and LS-C676024) and an immune checkpoint inhibitor (such as, for example, anti-CTLA-4 antibodies (including, but not limited to Tpilimumab or Tremelimumab), anti-PD1 antibodies (including, but not limited to Nivolumab, pembrolizumab, CT-011, and MK-3475), and/or anti-PD-L1 antibodies (including, but not limited to aitzolizumab, avelumab, durvalunab, MDX-1105, MPDL3280A, and MSB0010718C).

Also disclosed herein are methods of treating, inhibiting, reducing, decreasing, ameliorating, and/or preventing a cancer and/or metastasis (such as, for example melanoma) in a subject comprising administering to the subject the combination therapy of any preceding aspect. For example, disclosed herein are methods of treating, inhibiting, reducing, decreasing, ameliorating, and/or preventing a cancer and/or metastasis (such as, for example melanoma) in a subject comprising administering to the subject an anti-MARCO antibody (such as, for example, ED31, PY265, F3, Ab231046, AF7586, and LS-C6760243 and an immune checkpoint inhibitor (such as, for example, anti-CTLA-4 antibodies (including, but not limited to Ipilimumab or Tremelimumab), anti-PD1 antibodies (including, but not limited to Nivolumab, penbrolizumnab, CT-011, and MK-3475), and/or anti-PD-L1 antibodies (including, but not limited to atezolizumab, avelumab, durvalumab, MDX-1105, MPDL3280A, and MSB0010718C). In one aspect, the anti-MARCO antibody is administered intravenously and the immune checkpoint inhibitor is administered intraperitoneally.

In one aspect disclosed herein are methods of increasing the number of tumor infiltrating lymphocytes (TILs) and/or tumor-specific cytotoxic T lymphocyte (CTL) activity in a tumor microenvironment in a subject comprising administering to the subject the combination therapy of any preceding aspect. For example, disclosed herein are methods of increasing the number of tumor infiltrating lymphocytes (TILs) and/or tumor-specific cytotoxic T lymphocyte activity in a tumor microenvironment in a subject comprising administering to the subject an anti-MARCO antibody (such as, for example, ED31, PY265, F3, Ab231046, AF7586, and LS-C676024) and an immune checkpoint inhibitor (such as, for example, anti-CTLA-4 antibodies (including, but not limited to Ipilimumab or Tremelimumab), anti-PD1 antibodies (including, but not limited to Nivolumab, pembrolizumab, CT-011, and MK-3475), and/or anti-PD-L1 antibodies (including, but not limited to atezolizumab, avelumab, durvalumab. MDX-1105, MPDL3280A, and MSB0010718C). In one aspect, the anti-MARCO antibody is administered intravenously and the immune checkpoint inhibitor is administered intraperitoneally.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the effect of intravenous administration the anti-MARCO antibody ED31 on tumor growth is superior to intraperitoneal administration.

FIG. 2 shows that the combination of intravenous administration of ED31 and an anti-CTLA-4 administered intraperitoneally suppressive tumor growth to a much greater effect than either ED31 or an anti-CTLA-4 antibody alone.

FIG. 3 shows that survival of a tumor challenge increases with the combination of intravenous administration of ED31 and an anti-CTLA-4 administered intraperitoneally compared to either antibody alone.

FIG. 4A shows that the combination therapy of a immune checkpoint inhibitor and anti-MARCO significantly inhibited tumor growth. Shown is the tumor volume in B16F10 tumor bearing mice receiving anti-CTLA-4+ED31 combination therapy, anti-PD1+ED31 combination therapy, anti-CTLA-4 monotherapy, anti-PD1 monotherapy, anti-MARCO (ED31) monotherapy, or a control.

FIG. 4B shows survival curves B16F10 tumor bearing mice receiving anti-CTLA-4+ED31 combination therapy, anti-PD1+ED31 combination therapy, anti-CTLA-4 monotherapy, anti-PD1 monotherapy, anti-MARCO (ED31) monotherapy, or a control.

FIG. 5 shows that adding ED31 to anti-CTLA-4 increased the number of TILs in the TME.

FIG. 6 shows immunohistochemistry (IHC) staining of TME at day 10 post treatment showing adding ED31 to anti-CTLA-4 increased the number of TILs in the TME.

FIG. 7 shows the subpopulations of TILs in the TME at day 10 following treatment. Cells were stained with CD11b to sort myeloid cells and lymphoid cells and the characterized with further staining.

FIG. 8 shows the effect of ED31 on chemokine production was restricted to the TME. ED31(20 ug/mL) was pulsed ex-vivo with for 24 hours using 6-well plate (2*10⁶ cell/2 mL/well +/−ED31) for single-cell solutions of (A) Harvested tumor (=TME) from a tumor-bearing mouse (B16F10 implanted), (B) B16F1O (pure cell line culture), (C) Spleen(Sp), or (D) Lymph Node(LN). Chemokine concentrations of supernatants (2000g*10 min) were analyzed using multiplex assay kit (LEGENDplex, Biolegend).

FIG. 9 shows chemokine productivity of dendritic cells and macrophage.

FIG. 10 shows cytokine productivity of dendritic cells and macrophage.

FIG. 11 shows Tetramer assay for Ova-specific CTL detection in TME. After interventions (on Day7, 10, 13) for Ova+B16 implanted tumor-bearing mice, tumors were harvested on Day15. Then, single-cells of tumors were analyzed by Flow-cytometry using Ova-specific tetramer (MHC-1 tetramer complexes for SIINFEKL peptide, MBL) to detect Ova-specific CTL. For IFNγ analysis, single-cells were activated for 4-hr under Leukocyte Activation Cocktail (BD) before intracellular staining.

FIG. 12 shows that the Density of both cDC1 & cDC2 are significantly increased by Combination therapy. FIG. 12 also shows activated DCs are also increased in Combination therapy compared to αCTLA4 monotherapy.

FIG. 13 shows Treg inhibition. B16F10 tumor bearing mice were treated with either acti-CTLA-4 antibody+anti-MARCO antibody (ED31); anti-CTLA-4 antibody alone, ED31 alone, or a control and measured for the percentage of Tregs in the CD4 T cell population 15 days after administration of the treatment.

FIG. 14 shows the effect of the combination therapy on the CD8/Treg ratio in B16F10 tumor bearing mice following treatment. B16F10 tumor bearing mice were treated with either acti-CTLA-4 antibody+anti-MARCO antibody (ED31); anti-CTLA-4 antibody alone, ED31 alone, or a control and the number of CD8 T cells and T regs measured 15 days after administration of the treatment.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

As used herein “cytotoxic T lymphocyte (CTL) activity” refers to the activation of effector T cells, increase in CD8 T cell numbers, secretion of cytokines and chemokines by dendritic cells and macrophage that have influence T cell number and cytolytic function, and the secretion of effector molecules and cytokines by T cells, including, but not limited to Granzyme, IFN-γ, and TNF-0a.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts.

An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular combination therapy comprising an anti-MARCO antibody and immune checkpoint inhibitor is disclosed and discussed and a number of modifications that can be made to a number of molecules including the anti-MARCO antibody or immune checkpoint inhibitor are discussed, specifically contemplated is each and every combination and permutation of anti-MARCO antibody and immune checkpoint inhibitor and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Anti-CTLA4 Ab therapy is one of the standard therapies for various cancer, especially in human melanoma. However, the clinical efficacy of this single anti-CTLA4 therapy is known to be limited and only observed in less than 30% of the patients. The efficacy of cancer immunotherapy using dead tumor cell-pulsed dendritic cells (TP-DC) can be significantly increased by treating the TP-DC with an antibody to MARCO. Thus it was postulated herein that use of an anti-MARCO antibody could improve the efficacy of the immune checkpoint inhibitor.

MARCO is an integral membrane component composed of three 52-kDa monomers (Elomaa et al., Cell 80:603 (1995); Elomaa et al., J Biol Chem 273:4530 (1998)). Similar to the other SR-As, MARCO has a binding activity against Gram-positive and negative bacteria (van der Laan et al., J Immunol 162:939 (1999); van der Laan et al., Immunol Lett 57:203 (1997); Arredouani et al., J Immunol 175:6058 (2005); Elshourbagy et al., Eur J Biochem 267:919 (2000); Kraal et al., Microbes Infect 2: 313 (2000); Mukhopadhyay et al., Eur J Immunol 36:940 (2006)), modified low density lipoproteins (Elomaa et al., Cell 80:603 (1995); Kraal et al., Microbes Infect 2: 313 (2000)), as well as oxide and other particles (Arredouani et al., J Immunol 175:6058 (2005); Palecanda et al., J Exp Med 189:1497 (1999); Arredouani et al., J Exp Med 200:267 (2004)). MARCO expression was earlier identified in a subpopulation of macrophages in the marginal zone of the spleen and in the lymph node of the medullary cord (Elomaa et al., Cell 80:603 (1995)), and its expression was found to be up-regulated by bacterial LPS (van der Laan et al., Immunol Lett 57:203 (1997)) or systemic bacterial sepsis (van der Laan et al., J Immunol 162:939 (1999); van der Laan et al., Immunol Lett 57:203 (1997); Yoshimatsu et al., Int J Exp Pathol 85:335 (2004)). Further, MARCO is thought to play an important role in macrophage participation in some immune responses by mediating binding and phagocytosis, but also in the formation of lamellipodia-like structures and of dendritic processes. It has been reported that scavenger receptors have avid adherence to matrix molecules and to other cells (el Khoury et al., J Biol Chem 269:10197 (1994); Gowen et al., Matrix Biol 19:61 (2000); Karlsson et al., J Exp Med 198:333 (2003)). In an allergic airway inflammation model, scavenger receptor deficient innate DC revealed a higher level of migration into thoracic lymph nodes than control, wild-type DC (Arredouani et al., J Immunol 178:5912 (2007)).

Exemplary nucleic acid sequences for MARCO are NM-006770.3 for human, NM_010766.2 for mouse, and NM-001109011.1 for rat. Exemplary amino acid sequences for MARCO are NP-006761.1 for human, NP-034896.1 for mouse, and NP-001102481.1 for rat.

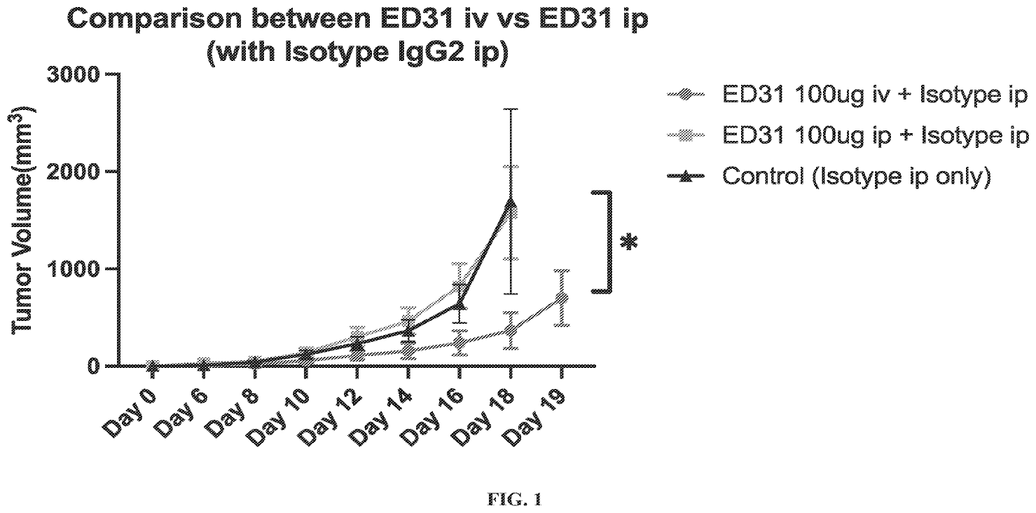

As shown herein adding intravenous ED31 to anti-CTLA4 therapy can show higher anti-tumor efficacy in murine melanoma, with more rapid tumor shrinkage and durable effect compared to the single anti-CTLA4 therapy (see FIGS. 1-3). Accordingly, in one aspect, disclosed herein are combination therapies comprising an anti-macrophage receptor with collagenous structure (MARCO) antibody (such as, for example, ED31, PY265, F3, Ab231046, AF7586, and LS-C676024) and an immune checkpoint inhibitor (such as, for example, anti-CTLA-4 antibodies (including, but not limited to Ipilimumab or Tremelimumab), anti-PD1 antibodies (including, but not limited to Nivolumab, perrbroli zumab, CT-011, and MK-3475), and/or anti-PD-L1 antibodies (including, but not limited to atezolizumab, avelumab, durvalurnab, MDX-1105, MPDL3280A, and MSB0010718C).

Anti-MARCO antibodies suitable for use in the present methods are known in the art and/or are commercially available. For example, anti-MARCO antibodies are commercially available from Hycult biotechnology by; Abcam; AbD Serotec; Abnova Corporation; Thermo Scientific; Acris Antibodies GmbH; BACHEM; BMA Biomedicals; Cell Sciences; GenWay Biotech, Inc.; LifeSpan BioSciences; Novus Biologicals; R&D Systems; Raybiotech, Inc.; and Santa Cruz Biotechnology, Inc. including, but not limited to ED31, PY265, F3, Ab231046, AF7586, and LS-C676024. The ED31 antibody used herein is available from Novus Biologicals and is described in, e.g., van der Laan et al. J. Immunol. 162: 939-947 (1999); and van der Laan et al., Immunol. Letters 57: 203-208 (1997). An anti-human MARCO antibody is described in Elomaa et al., J. Biol. Chem., 273(8):4530-4538 (1998). Alternatively, anti-MARCO antibodies can be made by the various methods known in the art.

In some embodiments, the anti-MARCO antibody or antigen-binding fragment thereof binds to the carboxyl-terminal cysteine-rich domain V of MARCO (residues 421-520 of the human MARCO polypeptide, see Elomaa et al., J. Biol. Chem., 273(8):4530-4538 (1998)). This domain is believed to be the ligand binding domain, and in some embodiments the anti-MARCO antibody blocks ligand binding to MARCO. Methods for determining whether an antibody binds to a particular region of a protein, and for determining whether antibody binding blocks ligand binding, are known in the art, e.g., peptide binding assays and competitive binding assays.

In some embodiments, the anti-MARCO antibody or antigen-binding fragment thereof increases or elicits MARCO signaling through p38, and decreases ERK signaling.

As used herein, immune checkpoint inhibitors comprise any antibody, small molecule, peptide, protein, siRNA, and/or shRNA that inhibits the interaction of a receptor and a ligand whose interaction inhibits immune responses. Examples of immune checkpoint inhibitors include, but are not limited to antibodies that block PD1 (such as, for example, Nivolumab (BMS-936558 or MDX1106), penbroizurmab, CT-011, MK-3475), PD-L1 (such as, for example, atezolizurab, avelurnah, durvalurnab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, B1754111, Sym022, FS118, MGD013, and Immutep).

### 1. Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with MARCO, PD1, PD-L1, CTLA-4, IDP-B7-H3, B7-H4, TIM3, TIGIT, BTLA, VISTA, or LAG3. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, sFv, scFv, and the like, including hybrid fragments. Thus, fragments of the antibodies that retain the ability to bind their specific antigens are provided. For example, fragments of antibodies which maintain MARCO, PD1, PD-L1, CTLA-4, IDP-B7-H3, B7-H4, TIM3, TIGIT, BTLA, VISTA, or LAG3 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### (4) Administration of Antibodies

Administration of the antibodies can be done as disclosed herein. Nucleic acid approaches for antibody delivery also exist. The broadly neutralizing anti-MARCO, PD1, PD-L1, CTLA-4, IDP-B7-H3, B7-H4, TIM3, TIGIT, BTLA, VISTA, and/or LAG3 antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### 2. Pharmaceutical Carriers/Delivery of Pharamceutical Products

As described above, the anti-MARCO antibodies and immune checkpoint inhibitors can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like.

Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. Methods of Using the Compositions

### 1. Method of Treating Cancer

In one aspect, disclosed herein are methods of treating, inhibiting, reducing, decreasing, ameliorating, and/or preventing a cancer and/or metastasis (such as, for example melanoma) in a subject comprising administering to the subject the combination therapy disclosed herein. Specifically, disclosed herein are methods of treating, inhibiting, reducing, decreasing, ameliorating, and/or preventing a cancer and/or metastasis (such as, for example melanoma) in a subject comprising administering to the subject an anti-MARCO antibody (such as, for example, ED31, PY265, F3, Ab231046, AF7586, and LS-C676024) and an immune checkpoint inhibitor (such as, for example, anti-CTLA-4 antibodies (including, but not limited to Ipilimumab or Tremelimumab), anti-PD1 antibodies (including, but not limited to Nivolumab, pernbrolizunab, CT-011, and MK-3475), and/or anti-PD-L1 antibodies (including, but not limited to atezolizumab, avelurnab, durvalunab, MDX-1105, MPDL3280A, and MSB0010718C).

Also disclosed herein are methods of increasing the number of tumor infiltrating lymphocytes (TILs) and/or tumor-specific cytotoxic T lymphocyte activity in a tumor microenvironment in a subject comprising administering to the subject any of the combination therapies disclosed herein. For example, disclosed herein are methods of increasing the number of tumor infiltrating lymphocytes (TILs) and/or tumor-specific cytotoxic T lymphocyte activity in a tumor microenvironment in a subject comprising administering to the subject an anti-MARCO antibody (such as, for example, ED31, PY265, F3, Ab231046, AF7586, and LS-C676024) and an immune checkpoint inhibitor (such as, for example, anti-CTLA-4 antibodies (including, but not limited to Ipilimumab or Tremelimumab), anti-PD1 antibodies (including, but not limited to Nivolumab, pembrolizumab, CT-011, and MK-3475), and/or anti-PD-L1 antibodies (including, but not limited to atezolizumab, avelumab, durvalumiab, MDX-1105, MPDL3280A, and MSB0010718C).

Anti-MARCO antibodies suitable for use in the present methods are known in the art and/or are commercially available. For example, anti-MARCO antibodies are commercially available from Hycult biotechnology by; Abcam; AbD Serotec; Abnova Corporation; Thermo Scientific; Acris Antibodies GmbH; BACHEM; BMA Biomedicals; Cell Sciences; GenWay Biotech, Inc.; LifeSpan BioSciences; Novus Biologicals; R&D Systems; Raybiotech, Inc.; and Santa Cruz Biotechnology, Inc. Such antibodies, include, but are note limited to ED31, PY265, F3, Ab231046, AF7586, and LS-C676024, The ED31 antibody used herein is available from Novus Biologicals and is described in, e.g., van der Laan et al. J. Immunol. 162: 939-947 (1999); and van der Laan et al., Immunol. Letters 57: 203-208 (1997). An anti-human MARCO antibody is described in Elomaa et al., J. Biol. Chem., 273(8):4530-4538 (1998). Alternatively, anti-MARCO antibodies can be made by the various methods known in the art.

In some embodiments, the anti-MARCO antibody or antigen-binding fragment thereof binds to the carboxyl-terminal cysteine-rich domain V of MARCO (residues 421-520 of the human MARCO polypeptide, see Elomaa et al., J. Biol. Chem., 273(8):4530-4538 (1998)). This domain is believed to be the ligand binding domain, and in some embodiments the anti-MARCO antibody blocks ligand binding to MARCO. Methods for determining whether an antibody binds to a particular region of a protein, and for determining whether antibody binding blocks ligand binding, are known in the art, e.g., peptide binding assays and competitive binding assays.

In some embodiments, the anti-MARCO antibody or antigen-binding fragment thereof increases or elicits MARCO signalling through p38, and decreases ERK signaling.

As noted above, the disclosed combination therapy comprises the administration of an immune checkpoint inhibitor. As used herein, immune checkpoint inhibitors comprise any antibody, small molecule, peptide, protein, siRNA, and/or shRNA that inhibits the interaction of a receptor and a ligand whose interaction inhibits immune responses. Examples of immune checkpoint inhibitors include, but are not limited to antibodies that block PD1 (such as, for example, Nivolumab (BMS-936558 or MDX1106), pembrolizumab, CT-011, MK-3475), PD-Li (such as, for example, atezolizunab, avelumab, durvalunab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, B1754111, Sym022, FS118, MGD013, and Immutep).

The disclosed combination therapy comprising anti-MARCO antibodies and immune checkpoint inhibitors can be used to treat any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers.

It is understood and herein contemplated that the route of administration can drastically effect the efficacy of the disclosed immunotherapy. As shown in FIGS. 1-3, administration of an anti-MARCO antibody intravenously has remarkably better efficacy than the same antibody and dosage administered intraperitoneally. The synergistic effect of an anti-MARCO antibody (such as, for example, ED31, PY265, F3, Ab231046, AF7586, and LS-C676024) and the immune checkpoint inhibitor (such as, for example, an anti-CTLA-4 antibody including, but not limited to Ipilimumab (MDX-010), Tremelimumab (CP-675,206)) can increase by administrative route. In one aspect, disclosed herein are methods of treating, inhibiting, reducing, decreasing, ameliorating, and/or preventing a cancer and/or metastasis (such as, for example melanoma) in a subject, wherein the anti-MARCO antibody (for example, ED31, PY265, F3, Ab231046, AF7586, and LS-C676024) is administered intravenously (i.v.) and the immune checkpoint inhibitor (such as, for example, an anti-CTLA-4 antibody including, but not limited to Ipilimumab (MDX-010), Tremelimumab (CP-675,206)) is administered intraperitoneally (i.p.). Also disclosed herein are methods of treating, inhibiting, reducing, decreasing, ameliorating, and/or preventing a cancer and/or metastasis (such as, for example melanoma) in a subject, wherein the anti-MARCO antibody (for example, ED31, PY265, F3, Ab231046, AF7586, and LS-C676024) is administered intravenously (i.v.) and the immune checkpoint inhibitor (such as, for example, an anti-CTLA-4 antibody including, but not limited to Ipilimumab (MDX-010), Tremelimumab (CP-675,206)) is also administered i.v.

When the same administrative route is utilized the anti-MARCO antibody (for example, ED31, PY265, F3, Ab231046, AF7586, and LS-C676024) and immune checkpoint inhibitor (such as, for example, an anti-CTLA-4 antibody including, but not limited to Ipilimumab (MDX-010), Tremelimumab (CP-675,206)) can be administered in the same composition (i.e., simultaneously). Administration by different routes can be concurrent or simultaneous. However, even if administered by the same route or a different route, the administration of the anti-MARCO antibody (for example, ED31, PY265, F3, Ab231046, AF7586, and LS-C676024) and immune checkpoint inhibitor (such as, for example, an anti-CTLA-4 antibody including, but not limited to Ipilimumab (MDX-010), Tremelimumab (CP-675,206)) can occur separated by seconds, minutes, hours, days, or even weeks. Additionally, the order of administration can include concurrent, simultaneous, or the anti-MARCO antibody being administered before or after the immune checkpoint inhibitor. In one aspect, administration of the anti-MARCO antibody occurs at least 5, 10, 15, 20, 25, 30, 35, 40, 45, 50, 55, 60, 65, 70, 75, 80, 85, 90 seconds 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90, 105, 120, 130, 140, 150 minutes, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72 hours, 4, 5, 6, 7, 8, 9, 10, 11, 12, 13, 14, 15, 16, 17, 18, 19, 20, 21, 22, 23, 24, 25, 26, 27, 28, 29, 30, 31, 35, 42, 49, 56, 59, 60, 61, 62, 63, 70, 77, 84, 90 days, 3, 4, 5, 6, 7, 8, 9, 10, 11, 12, 13, 14, 15, 16, 17, or 18 months before administration of the immune checkpoint inhibitor. Alternatively, administration of the anti-MARCO antibody occurs at least 5, 10, 15, 20, 25, 30, 35, 40, 45, 50, 55, 60, 65, 70, 75, 80, 85.90 seconds 2,3,4,5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90, 105, 120, 130, 140, 150 minutes, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72 hours, 4, 5, 6, 7, 8, 9, 10, 11, 12, 13, 14, 15, 16, 17, 18, 19, 20, 21, 22, 23, 24, 25, 26, 27, 28, 29, 30, 31, 35, 42, 49, 56, 59, 60, 61, 62, 63, 70, 77, 84, 90 days, 3, 4, 5, 6, 7, 8, 9, 10, 11, 12, 13, 14, 15, 16, 17, or 18 months after administration of the immune checkpoint inhibitor.

While it is desired for a single administration of the anti-MARCO antibody (for example, ED31, PY265, F3, Ab231046, AF7586, and LS-C676024) and immune checkpoint inhibitor (such as, for example, an anti-CTLA-4 antibody including, but not limited to Ipilimumab (MDX-010), Tremelimumab (CP-675,206)) to be efficacious, it is understood and herein contemplated that multiple administrations of either the anti-MARCO antibody (for example, ED31, PY265, F3, Ab231046, AF7586, and LS-C676024) and immune checkpoint inhibitor (such as, for example, an anti-CTLA-4 antibody including, but not limited to Ipilimumab (MDX-010) or both may be needed to achieve an efficacious result. Accordingly, disclosed herein are methods of treating, inhibiting, reducing, decreasing, ameliorating, and/or preventing a cancer and/or metastasis (such as, for example melanoma) in a subject, wherein the anti-MARCO antibody (for example, ED31, PY265, F3, Ab231046, AF7586, and LS-C676024) wherein the anti-MARCO antibody and/or the immune checkpoint inhibitor is administered 1, 2, 3, 4, 5, 6, 7, 8 9, or 10 times.

Where multiple administrations of the anti-MARCO antibody and/or the immune checkpoint inhibitor is utilized, administration of a subsequent dose can occur 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 42, 49, 56, 59, 60, 61, 62, 63, 70, 77, 84, 90 days, 3, 4, 5, 6, 7, 8, 9, 10, 11, 12, 13, 14, 15, 16, 17, or 18 months after administration of the preceding dose.

In one aspect, the anti-MARCO antibody is administered intravenously and the immune checkpoint inhibitor is administered intraperitoneally.

It is understood and herein contemplated that the disclosed combination therapies can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil-Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (lbrutinib), lmfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine 1131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride , Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil-Topical), Topotecan Hydrochloride, Toremifene, Torisel (Temsirolimus), Tositumomab and Iodine 1131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

### D. Examples

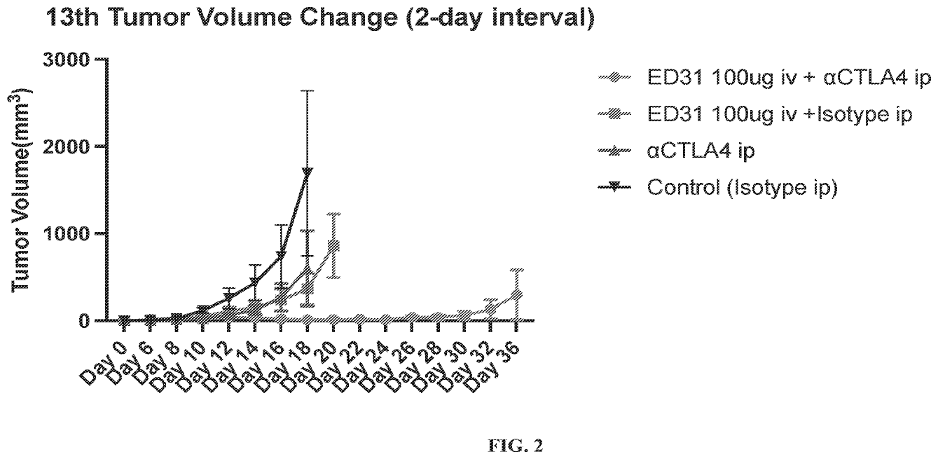

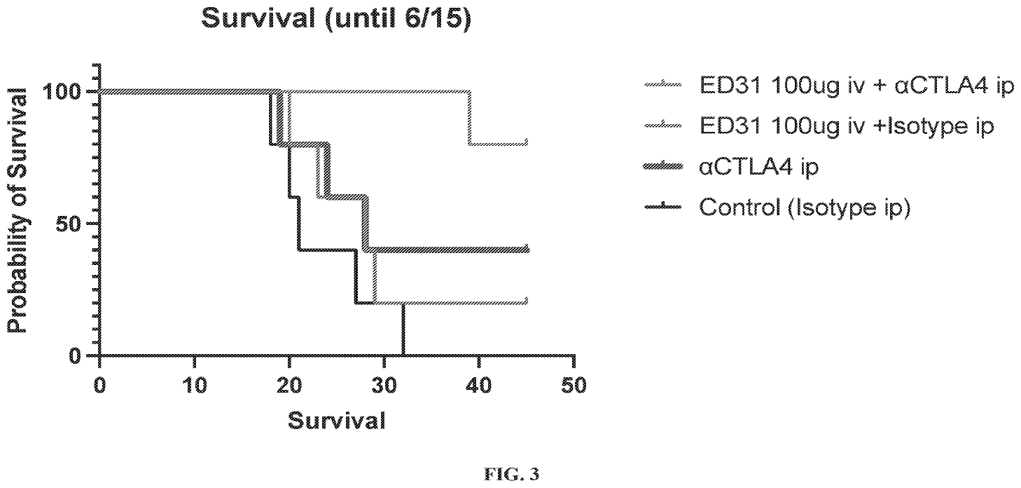

To investigate the effect of anti-MARCO antibodies on immune checkpoint inhibitors, we first investigated whether the route of administration had any effect on the performance of the anti-MARCO antibody ED31 on tumor volume. We found that administration of ED31 intravenously significantly outperformed administration intraperitoneally (FIG. 1). Next, we investigated the effect of ED31 in combination with anti-CTLA-4 antibodies on tumor volume (FIG. 2) and survival (FIG. 3), relative to either anti-MARCO antibody or anti-CTLA-4 antibody alone. We found a significant improvement in reduction in tumor volume and increased survival in the combination relative to either anti-MARCO antibody or anti-CTLA4 antibody alone.

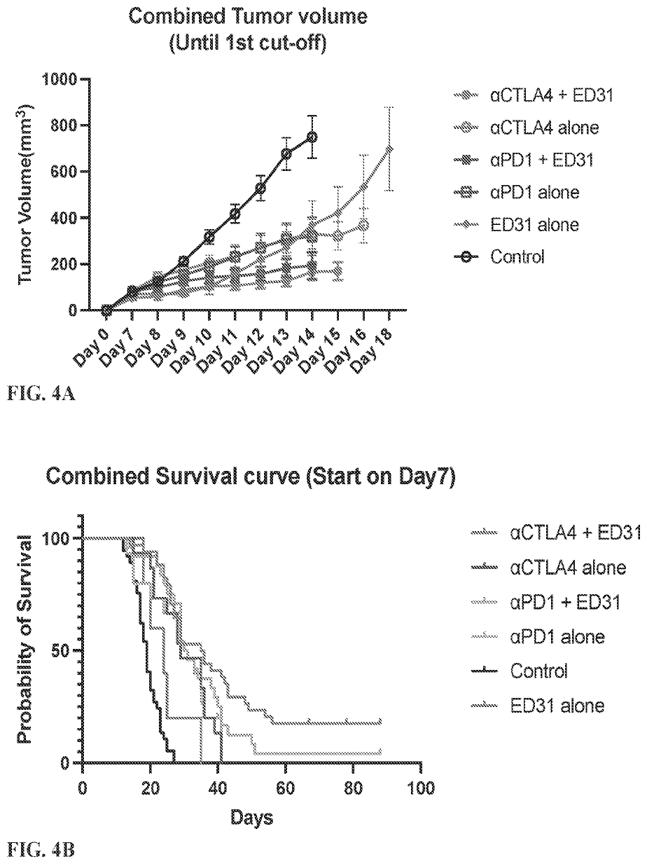

We then investigated whether the effect of the anti-MARCO antibody observed in combination with anti-CTLA-4 antibody would also be observed with other checkpoint inhibitors. Thus, we administered anti-MARCO with or without the combination of anti-CTLA-4 or anti-PD1 antibody and measured tumor volume (FIG. 4A) and survival (FIG. 4B). We observed that the combination of anti-MARCO and anti-PD1 outperformed either anti-MARCO or anti-PD1 antibody alone, though not as well as the combination of anti-CTLA-4 antibody and anti-MARCO antibody.

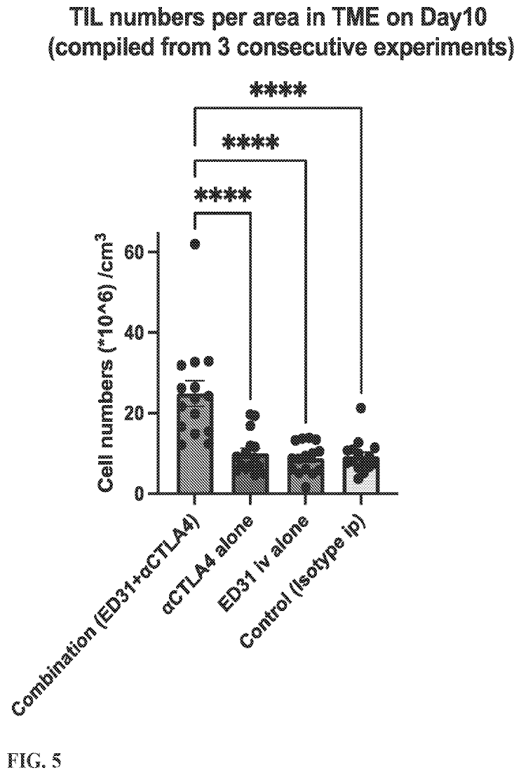

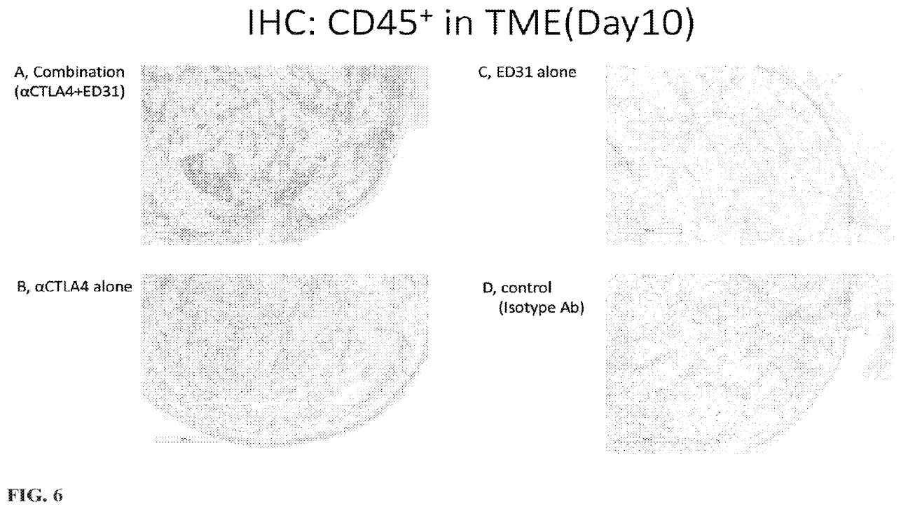

To determine the cause of the positive effects on the combination of anti-MARCO and immune checkpoint inhibitor, we measured the number of TILs in the tumor microenvironment (TME) to days after administration (FIGS. 5 and 6) following administration of the combination of anti-CTLA-4 antibody and anti-MARCO antibody. We observed a 2-3 fold increase in total numbers of TILs with the combination treatment relative to either anti-MARCO or anti-CTLA-4 alone which showed no increase relative to untreated controls. Thus, the combination treatment significantly impacts the attraction of TILs to the TME.

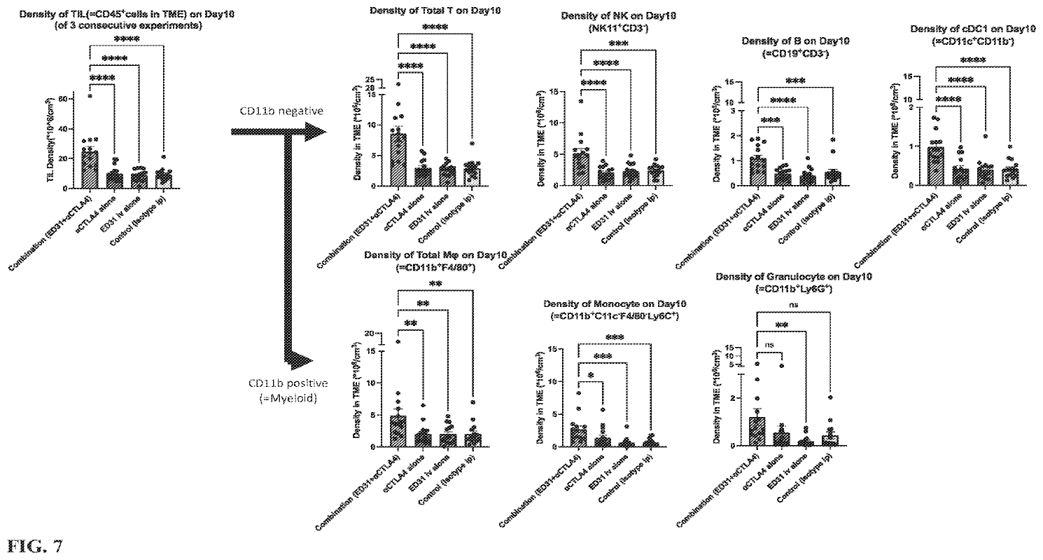

Looking into the composition of the TILs that were in the TME following administration of the combination therapy, we stained for CD11b (to distinguish myeloid from lymphoid cells) and characterized the cell types (FIG. 7). We observed an increase in all lymphoid populations. Specifically, we measured at least a two-fold increase in NK cells, B cells, and cDC1 cells and a four-fold increase in T cells. Similarly, we observed a two-fold increase in macrophage, monocytes, and granulocytes.

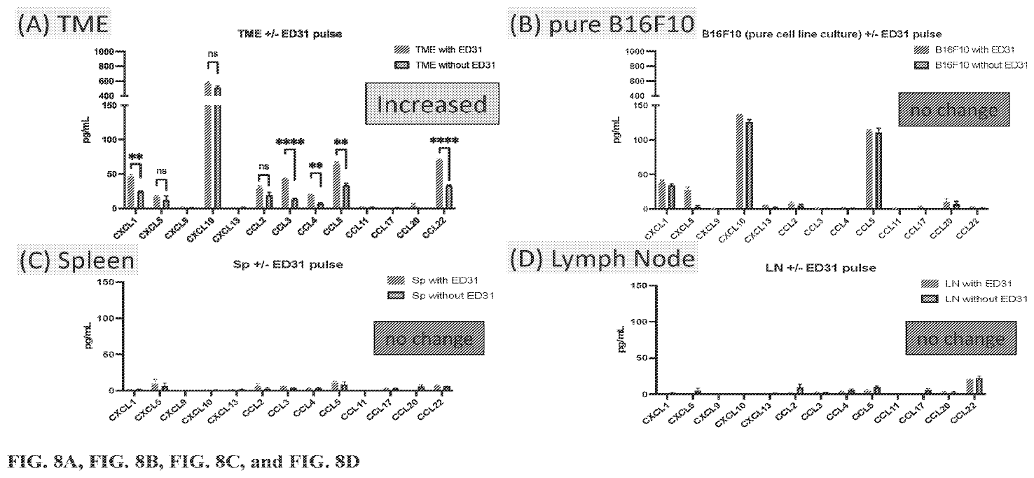

We then investigated the underlying mechanism of the TIL increase in the anti-MARCO/anti-CTLA-4 combination therapy. To understand the mechanism, B16F10 tumor bearing mice received monotherapy of anti-MARCO antibody (ED31) or control treatment. Lymph nodes, spleens and tumor tissue was harvested after 15 days and measured for various chemokines. Looking at lymph nodes and spleens, no change in any chemokine was observed in the mice receiving anti-MARCO compared with untreated mice. However, in the TME, there was a significant increase in at least CCL22, CCL5, CXCL1, and CCL3. Mice receiving treatment also showed an increase in CXCL10, CXCL5, and CCL2, though the significance of these increases was not established (FIG. 8).

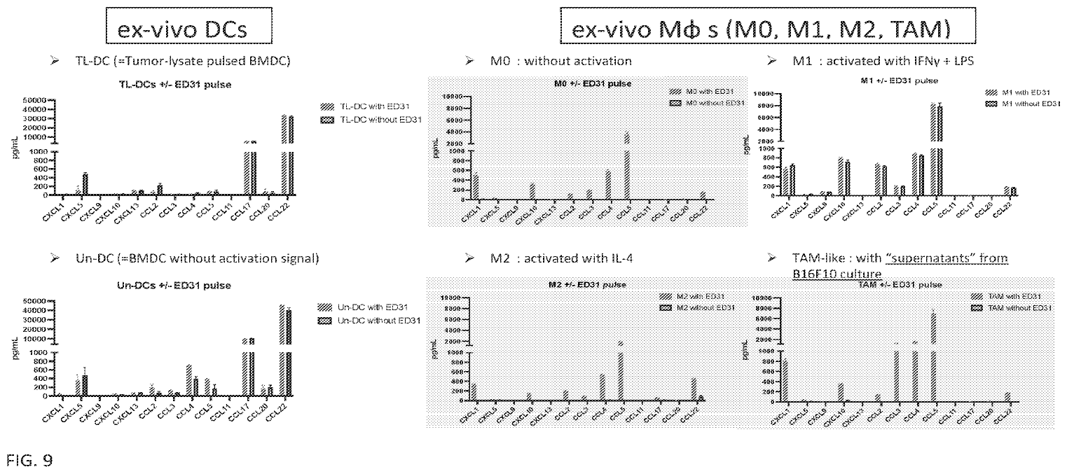

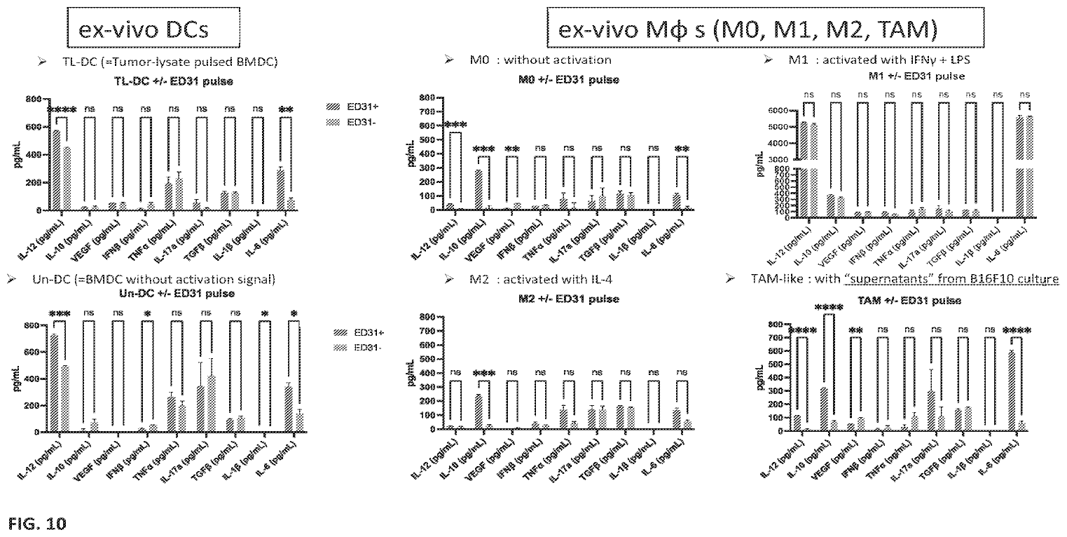

Next we investigated the effect of ED31 on Myeloid cells in terms of chemokine and cytokine productivity. Here, bone marrow from B16F10 tumor bearing mice either receiving ED31 treatment or a control was harvested and cultured for seven days with M-CSF. Cultured cells were then either harvested with no activation (to measure MO), activated with IFN-γ+LPS (to measure M1), IL-4 (to measure M2), or activated with supernatants from a tumor cell line (to measure TAM-like phenotype). Measuring chemokines (FIG. 9), we observed an increase kin several chemokines in treated mice at the MO, M2, and TAM-like phenotypes, specifically at CXCL1, CXCL10, CCL2, CCL3, CCL4, CC15, and CCL22. Similar increases were not observed in M1 macrophage. Looking at cytokine expression (FIG. 10), IL-10 was increased in MO, M2, and TAM-like macrophage from mice receiving ED31, with TAM-Like macrophage also having increases in IL-6, IL-12, and IL-17a production.

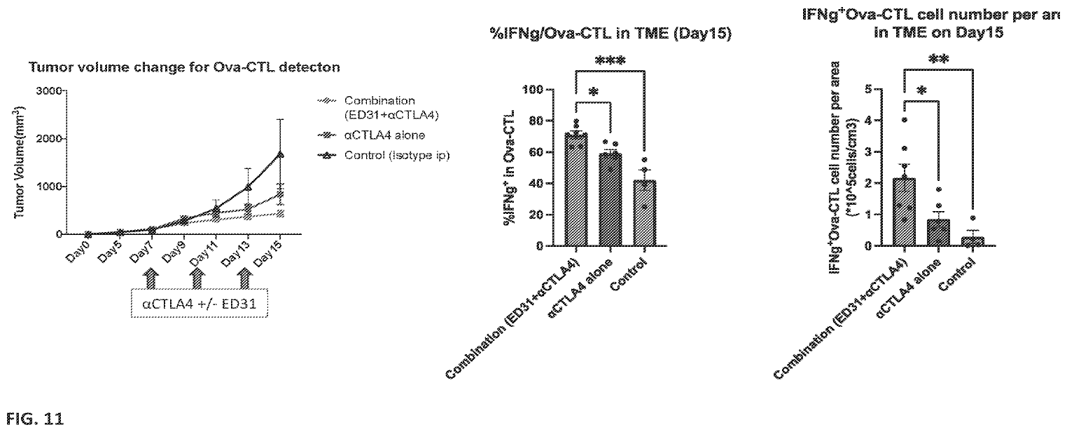

We next investigated the effect of the combination therapy of anti-MRCO antibody (ED31) and anti-CTLA-4 antibody on cytotoxic T lymphocytes. We measured tumor volume, CTL number, and IFN-7 (cytolytic activity) in harvested T cells (FIG. 11). We observed a significant decrease in tumor volume in mice receiving the combination therapy alone with an increase in the number of CTL in the TME and an increase in CTL activation. Thus, adding ED31 to anti-CTLA4 can not only activate Tumor-specific CTLs (capacity of IFNγ production), but also increase Tumor-specific CTL cell number per area in TME.

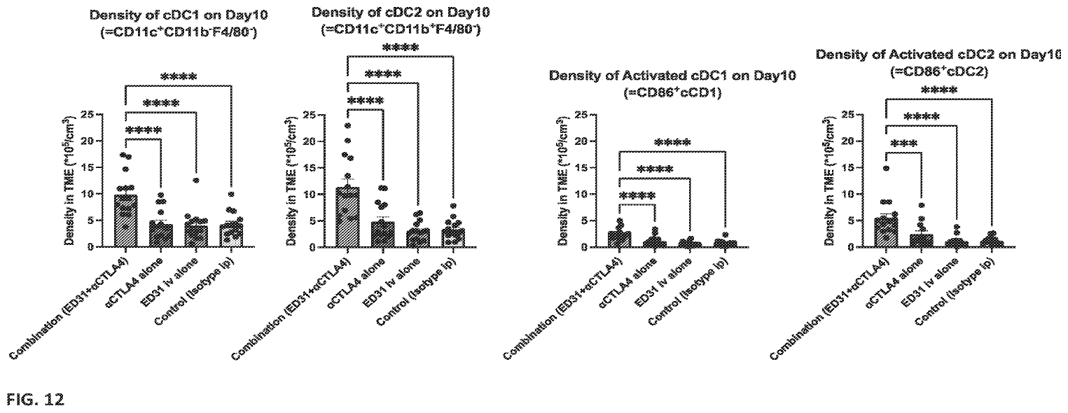

Further characterizing the TME, we measured the density of both naïve and activated cDC1 and cDC2 populations following combination treatment (FIG. 12). We observed an overall increase in both cDC1 and cDC2 for both the naïve and activated cells following anti-CTLA-4 and anti-MARCO (ED1) combination treatment compared to monotherapy of either.

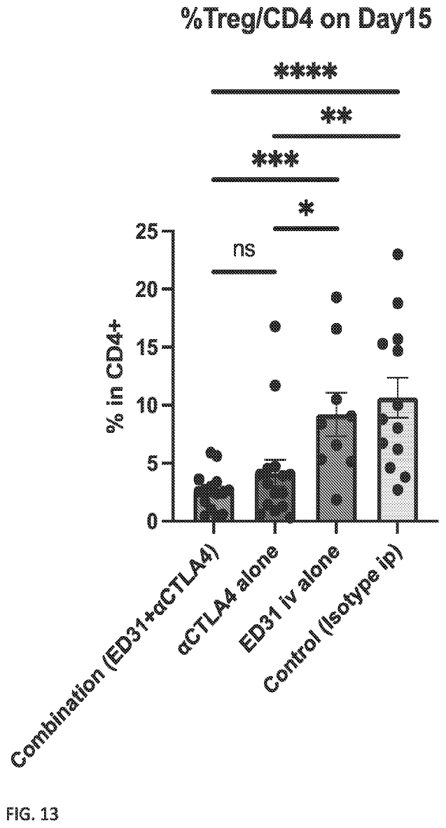

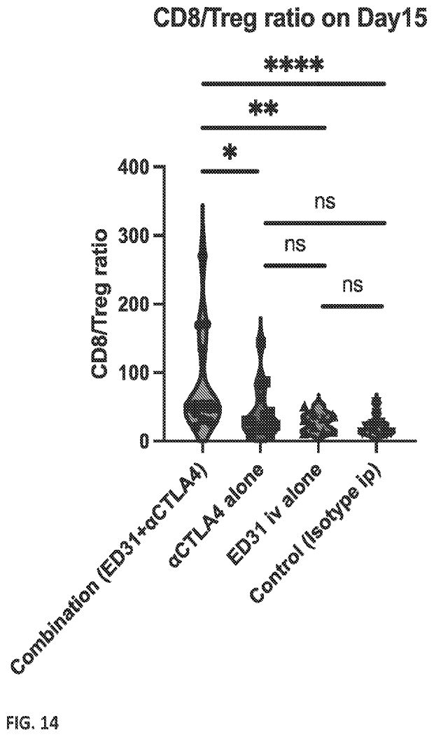

Lastly, we measured the impact of Trefs on T cells in the microenvironment following administration of the combination of ED31 and anti-CTLA-4. We found the combination therapy had a greater reduction in the percentage of Treg inhibition by day 15 relative to either antibody alone (FIG. 13). Similarly, we observed that the ratio of CD8T cells to Tregs increased in the combination treatment significantly more than either therapy alone (FIG. 14).

1. A combination therapy comprising a) an anti-macrophage receptor with collagenous structure (MARCO) antibody and b) an immune checkpoint inhibitor.

2. The combination therapy of claim 1, wherein the anti-MARCO antibody is selected from the group consisting of ED31, PY265, F3, Ab231046, AF7586, and LS-C676024.

3. The combination therapy of claim 1, wherein the immune checkpoint inhibitor comprises an anti-CTLA-4 antibody, an anti-PD1 antibody, or an anti-PD-L1 antibody.

4. The combination therapy of claim 3, wherein the anti-CTLA-4 antibody comprises Ipilimumab or Tremelimumab.

5. The combination therapy of claim 3, wherein the anti-PD1 antibody comprises Nivolumab, pembrolizumab, CT-011, or MK-3475.

6. The combination therapy of claim 3, wherein the anti-PD-L1 antibody comprises atezolizumab, avelumab, durvalumab, MDX-1105, MPDL3280A, or MSB0010718C.

7. (canceled)

8. A method of treating a cancer in a subject comprising administering to the subject a)_an anti-MARCO antibody and b) an immune checkpoint inhibitor.

9. The method of claim 8, wherein the anti-MARCO antibody is selected from the group consisting of ED31, PY265, F3, Ab231046, AF7586, and LS-C676024.

10. The method claim 8, wherein the immune checkpoint inhibitor comprises an anti-CTLA-4 antibody, an anti-PD1 antibody, or an anti-PD-L1 antibody.

11. The method of claim 10, wherein the anti-CTLA-4 antibody comprises Ipilimumab or Tremelimumab.

12. The method of claim 10, wherein the anti-PD1 antibody comprises Nivolumab, pembrolizumab, CT-011, or MK-3475.

13. The method of claim 10, wherein the anti-PD-L1 antibody comprises atezolizumab, avelumab, durvalumab, MDX-1105, MPDL3280A, or MSB0010718C.

14. The method of claim 8, wherein the anti-MARCO antibody is administered intravenously.

15. The method of claim 8, wherein the immune checkpoint inhibitor is administered intraperitoneally.

16. The method of claim 8, wherein the cancer is melanoma.

17. (canceled)

18. A method of increasing the number of tumor infiltrating lymphocytes (TILs) and/or tumor-specific cytotoxic T lymphocyte activity in a tumor microenvironment in a subject comprising administering to the subject a) an anti-MARCO antibody and b an immune checkpoint inhibitor.

19. The method of claim 18, wherein the anti-MARCO antibody is selected from the group consisting of ED31, PY265, F3, Ab231046, AF7586, and LS-C676024.

20. The method of claim 18, wherein the immune checkpoint inhibitor comprises an anti-CTLA-4 antibody, an anti-PD1 antibody, or an anti-PD-L1 antibody.

21. The method of claim 20, wherein the anti-CTLA-4 antibody comprises Ipilimumab or Tremelimumab.

22. The method of claim 20, wherein the anti-PD1 antibody comprises Nivolumab, pembrolizumab, CT-011, or MK-3475.

23. The method of claim 20, wherein the anti-PD-L1 antibody comprises atezolizumab, avelumab, durvalumab, MDX-1105, MPDL3280A, or MSB0010718C.

24. The method of claim 18, wherein the anti-MARCO antibody is administered intravenously.

25. The method of claim 18, wherein the immune checkpoint inhibitor is administered intraperitoneally.

26. The method of claim 18, wherein the subject has a cancer, and wherein the cancer is melanoma.
